(19) United States
(12) Patent Application Publication
(10) Pub. No.: US 2006/0241728 A1
(43) Pub. Date: Oct. 26, 2006

(54) Control equipment for holding a laparoscopic probe

(76) Inventor: Deshpande Suresh Vamanrao, Maharashtra (IN)

Correspondence Address: HEDMAN & COSTIGAN P.C., 1185 AVENUE OF THE AMERICAS, NEW YORK, NY, 10036, US

(21) Appl. No.: 11/351,419

(22) Filed: Feb. 10, 2006

(30) Foreign Application Priority Data
Feb. 11, 2005 (IN) 151/MUM/2005

(51) Int. Cl. A61N 5/06

(52) U.S. Cl. 607/92

(57) ABSTRACT

A control equipment is provided for precisely positioning the tip of a laparoscopic probe in a body cavity. The control equipment consists of, a housing adapted to be arcuately movable about a vertical axis; a post extending from said housing, an arm fitted in proximity to the apex of the operative top end of said post, and a clamp adapted to be fitted to said free end and adapted to hold the probe. Drive mechanisms are provided to displace said housing, post and arm. Foot operated pedals drive the drive mechanism to position the tip of said probe in its operative configuration within the body cavity.

### FIELD OF THE INVENTION

This invention relates to control equipment for endoscopic surgery.

Particularly, this invention relates to control equipment for performing minimally invasive endoscopic surgical procedures, typically laparoscopic surgical procedures.

### BACKGROUND OF THE INVENTION

### Introduction

Performing surgical procedures on the human body is a very critical and complicated task, which can be undertaken only by those surgeons who are experienced and have a thorough understanding of the human anatomy and procedures relating to the same. The human body has so many organs that surgeons specialized only in one particular human organ performs such surgeries. This fact becomes more important when it comes to surgeries related to the smaller and more complicated parts of the human anatomy.

Traditional approaches to the surgical procedures involved the use of longer incisions so that surgeons can have a better view of the organ that they are working on. However longer incisions led to a number of problems such as:
(i) longer incisions require more time to heal; and
(ii) more post operative care is necessary.

Also the same surgical apparatus were used for different kind of surgeries. The use of bulky surgical equipment causes trauma to the organs surrounding the organ being operated, leading to internal bleeding and further complications.

Thus there is a need for more precise and specialized equipment for the purpose of performing surgeries, typically surgeries for treating diseased organs present in the abdominal cavity and the pelvic region. It was necessary that while operating on the organs in said region of the body the organ were distinctly visible, as one wrong step could prove fatal.

Hence laparoscopic surgeries have been introduced. Laparoscopic surgeries make use of a laparoscope, which consists of:
(i) a fibre optic system to illuminate the operative site,
(ii) a lens system to view the operative site that is usually connected to a video camera; and
(iii) a channel to allow access for intervention using long, thin instruments.

A laparoscope delivers the image of a surgical site through a series of lenses stacked within a rod that contains optical fibers for transmission of light to illuminate the site. Typically, laparoscopes are either 5 or 10 mm in diameter, but a more universal 2.7 mm diameter scope can also be used for laparoscopy. The light transmitted and size of the image observed is directly proportional to the diameter of the laparoscope. Laparoscopes can be zero degree viewing laparoscope such that viewing is “straight-on” and the image is centered on the axis of the scope. A 30-degree viewing laparoscopes permits viewing from the side, a useful view when working in many remote body regions. These 30 degree scopes are used for thoracoscopy, to examine the cranial and caudal regions of the abdomen, for rhinoscopy, cystoscopy, and otoscopy.

Other instruments, which may also be used during laparoscopic surgery are:
(i) an insufflator machine for inflating the body cavity with CO2 gas, which is designed to deliver the gas at a desired flow rate, and to measure the absolute pressure generated within the body cavity being filled.
(ii) a video monitor for the purpose of displaying the images that are observed through the lens and captured by the camera.

Through an incision a trocar cannula is introduced into the body cavity of the patient. The trocar is then removed and a laparoscopic probe is introduced into the body of the patient through the cannula. Size of the incision is in the range of 1 cm to 5 cm, through the incision the laparoscope is slid into the body of the patient. The size of the incision required for surgery is small and hence it is also known as key hole surgery. When used in the abdominal wall for laparoscopy, these trocars must be able to retain carbon dioxide within the abdomen. For thoracoscopy, the trocars only need to serve as portals as the lung elasticity will retract the lung sufficiently to permit examination of the chest.

Almost every organ in the human body has become accessible to the surgeon's camera and scalpel. Gallstones are now being removed with the gallbladder by laparoscopic surgery in over 90% of patients presenting with this disorder. Instead of months of bed rest and limited activities, which was associated with the old method of removing the gallbladder, patients now resume their normal activities in several weeks.

Many other organs are also easily accessed in a similar manner. These include the stomach, intestines, pancreas and spleen, kidneys, reproductive organs and the like. Operations have also been developed for diseases of the bladder and the prostate in men.

Procedure of the laparoscopic surgery can be summarized as follows:
(i) the patient is helped onto the operating table.
(ii) the anesthesiologist injects the anesthetic through the IV.
(iii) once the patient looses consciousness the nurses cleans the abdomen with antibacterial soap.
(iv) the surgeon then places a small needle just below the belly button (umbilicus) and insert the needle into the abdominal cavity of the patient. This needle is connected to sterile tubing, and carbon dioxide is passed into the abdominal cavity through the tubing. The gas lifts the abdominal wall away from the organs below. This space gives the surgeon a better view of the abdominal cavity once the laparoscope is in place.
(v) a small incision is made near the belly button. The laparoscope is introduced into the body cavity of the patient through this incision. The image the surgeon sees in the laparoscope is projected onto video monitors placed near the operating table.
(vi) Before starting the surgery, the surgeon takes a thorough look at the abdominal cavity to make sure that laparoscopy is safe for the patient. Some reasons why laparoscopy might not be done include multiple adhesions, infection, or any widespread abdominal disease.
(vii) If the surgeon decides that laparoscopic surgery can be safely performed, additional incisions are made, which gives the surgeon access to the abdominal cavity. The number and location of the incisions depend on the type of surgery being performed.
(viii) If required one of these small incisions might be enlarged to enable the surgeon to remove the diseased section of the organ or the organ itself.
(ix) the surgeon then performs the required corrective procedure for the particular organ;
(x) after the corrective procedure the surgeon checks for traces of internal bleeding, the abdominal cavity is then rinsed, the carbon-di-oxide gas is released from the abdomen and incisions are stitched.
(xi) patient is then transferred to the recovery room.

The advantages of this method of operating are several.
(i) the overall trauma to the skin and muscles is reduced.
(ii) post operative pain is less—allowing patients to get out of bed sooner.
(iii) patients are able to walk and move around within a few short hours following their operations.
(iv) reduced infection rate as delicate tissues are not exposed to the air of the operating room over long periods of time as they are when the body is wide open in traditional operations.
(v) Video magnification also offers surgeons better exposure of the diseased organ and its surrounding vessels and nerves and
(vi) delicate maneuvers can he performed to protect these vital structures during the removal or repair of target organs.

A few disadvantages of laparoscopy include:
(i) the equipment used for the operation is expensive;
(ii) surgeons require special training for performing such an operation using the available means; and
(iii) surgeons who are brilliant in open techniques need special training to transfer their excellent surgical skills to the video monitor and display.

The need for additional training is because laparoscopic surgeons leave the familiar territory of a three dimensional operating field to working on a two dimensional flat video display. The shift is a critical one, and requires some degree of practice moving around long laparoscopic instruments while handling delicate tissues. Despite these temporary disadvantages, with the proper training, surgeons are able to adapt to this means of operating.

A few common illnesses diagnosed through laparoscopy are endometriosis, pelvic inflammatory disease, ectopic pregnancy, ovarian cysts, appendicitis, gallbladder stones, kidney stones and the like.

Initially the laparoscope was used only for viewing purposes i.e the doctor's assistant would hold the laparoscope and move it manually. The surgeon would then place his eye at the camera lens and perform the operation. Subsequently the images from the laparoscopic camera were taken to a remotely located viewing device such as a monitor, but the laparoscope was still moved by the surgeons assistant. The surgeon would direct the assistant to move the laparoscope for his viewing. This created a problem of timing and accuracy because of miss communication between the surgeon and his assistant. As a result several equipments were designed for the surgeon to manipulate the tip of the laparoscopic probe within a body cavity. Some of these equipments are discussed below.

U.S. Pat. No. 6,070,584 discloses a support structure for fixing a laparoscope in a predetermined position relative to a patient on an operating table. The support structure comprises a vertical support arm, which is displaceable on a horizontal support arm directed transversely to the operating table. The disclosed support structure positions the laparoscope in a predetermined position thus restricting freedom of movement, which would be essential for a surgeon

U.S. Pat. No. 6,530,880 discloses an apparatus, which supports an endoscope for viewing a surgical site in a patient during surgery on the patient. The apparatus includes a base plate, a part is adapted to be fixed to the endoscope, and a screw mechanism. The disclosed apparatus restricts the movement of the endoscope thus it is difficult to change the position of the endoscope whenever required.

U.S Patent Application No. 20020165524 discloses pivot port that can provide a pivot point for a surgical instrument. The pivot port may be held in a stationary position by a support arm assembly that is attached to a table. The disclosed pivot port makes use of many joints thus making the system very complex and expensive.

U.S Patent Application No. 20040111183 discloses a robot system for use in surgical procedures has two movable arms each carried on a wheeled base with each arm having a six of degrees of freedom of movement and an end effector which can be rolled about its axis and an actuator which can slide along the axis for operating different tools adapted to be supported by the effector. The disclosed system is a very complex system involving many moving parts. The system requires a console for maneuvering the arms thus making it heavier and more expensive.

U.S Patent Application No. 20040261179 discloses methods, especially for minimally invasive surgery, and more particularly provides structures and techniques for aligning a robotic surgery system with a desired surgical site. The present invention describes techniques for mounting, configuring and arranging set-up arms for the surgical manipulators and endoscope drive mechanisms of a telesurgical system within an operating theater. Such a system and a method are difficult to implement and the use of ceiling mounted equipment requires special arrangements, thus making the overall system very expensive.

This invention seeks to overcome the limitations of the prior art.

The object of the invention is to provide a simple, efficient and inexpensive device for the purpose of holding the laparoscope.

Another object of this invention is to provide an automated precision guidance system for guiding the laparoscope during a laparoscopic surgery.

Another object of this invention is to provide a system having reduced complexity in design and less number of moving parts thus reducing the overall cost of the system.

### SUMMARY OF THE INVENTION

According to this invention there is provided a control equipment, for holding a laparoscopic probe and precisely positioning the tip of said probe in its operative configuration within a body cavity said control equipment comprising:
(i) a housing adapted to be arcuately movable about a vertical axis in a first plane in its operative configuration;
(ii) a post extending from said housing, angularly displaceable in a second plane being perpendicular to said first plane;
(iii)an arn fitted in proximity to the apex of the operative top end of said post, said arm being angularly displaceable in a third plane being mutually perpendicular to said first and second planes and defining a free end;
(iv)a clamp adapted to be fitted to said free end and adapted to hold the probe of a laparoscope in position;
(v) discrete drive mechanisms adapted to displace said housing, post and arm in respective first, second and third planes; and
(vi)control means adapted to drive each of said discrete drive mechanism means to position the tip of said probe in its operative configuration within a body cavity.

Typically, said control means are a plurality of discrete foot operated means adapted to drive each of said discrete drive mechanisms.

Typically, said housing is adapted to be rotatably fixed to a pedestal having wheels for moving said control equipment.

Typically, said discrete drive mechanism means are housed within said housing.

Typically, a first fitment means is provided between said post and said housing in the form of a cradle support joint.

Typically, a second fitment means is provided between said arm and said post in the form of a swivel joint.

According to another aspect of this invention, there is provided a method for moving the tip of a laparoscopic probe within a body cavity using a control equipment having an arcuately movable housing, a post extending therefrom, an arm in proximity to the apex of said post in its operative configuration and a clamp fitted to one end of the arm for the purpose of holding a laparoscope, said method comprising:
(a) inserting said laparoscopic probe through a cannula into a body cavity said junction of body cavity and the cannula acting as a fulcrum point:
(b) moving said probe in and out of a body cavity by arcuately moving housing of said control equipment about its vertical axis in its operative configuration in a first plane;
(c) moving the viewing tip of said laparoscopic probe operatively in the left and right direction at said fulcrum point by angularly displacing said post along an operatively vertical axis in a second plane perpendicular to said first plane;
(d) moving the viewing tip of said probe operatively up and down by swiveling said arm along an axis defined by a plane parallel to a horizontal axis and being mutually perpendicular to said first and second planes; and
(e) independently controlling said movements of the laparoscopic probe in said planes by means of remotely located control means to accurately position the viewing tip of said laparoscopic probe at a desired point within said body cavity.

Typically, said angular left and right movement of laparoscopic probe at said fulcrum point respectively corresponds to a right and left movement of the tip of the laparoscopic probe within said body cavity.

Typically, said upward and downward movement of the laparoscopic probe at said fulcrum point respectively corresponds to a downward and upward movement of the tip of the laparoscopic probe within said body cavity

In accordance with one practical embodiment of such a device this invention envisages the use of foot operated pedals for the purpose of moving the various components and the housing of the control equipment.

### BRIEF DESCRIPTION OF THE ACCOMPANYING DRAWINGS

The invention will be described in detail with reference to a preferred embodiment. Reference to this embodiment does not limit the scope of the invention.

In the accompanying drawings:

FIG. 1 illustrates the directions along which the viewing tip of the laparoscopic probe can be moved;

FIG. 2 illustrates the laparoscopic surgical equipment;

FIG. 3 illustrates an overview of the control equipment in its operative configuration;

FIG. 4 illustrates a schematic block diagram of the components used to displace the housing, post and the arm of the control equipment; and

FIG. 5 illustrates a real time image of the use of the laparoscopic equipment.

### DETAILED DESCRIPTION OF THE DRAWINGS

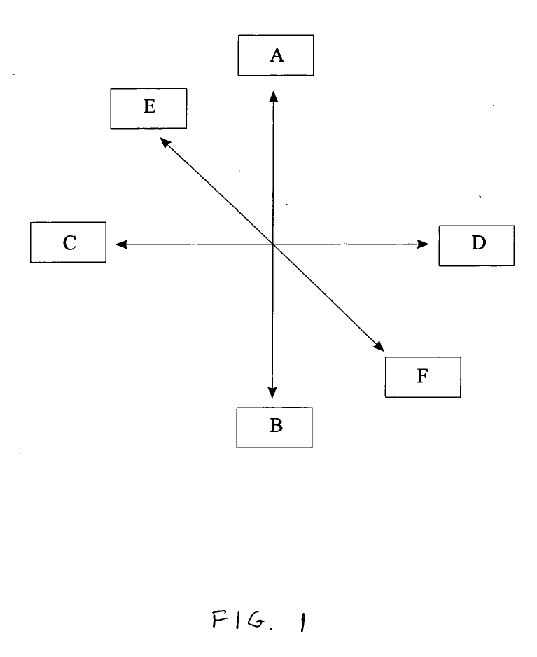

The invention will now be explained with reference to FIGS. 1 to 5 of the accompanying drawings.

FIG. 1 illustrates the directions along which the viewing tip of the laparoscopic probe can be moved. In the process of performing the laparoscopic surgical procedure it is essential that the surgeon has a wide range of movement, which helps better visualization of the organ being operated. In accordance with one preferred embodiment of such an equipment this invention provides movement of a laparoscope in the directions indicated by the reference alphabets (A), (B), (C), (D), (E) and (F). The laparoscope can be moved in the upward and downward direction indicated by (A) and (B) respectively. Numerals (C) and (D) indicate the movement of the laparoscope in the left and the right directions respectively. It is also essential that the laparoscope be moved in and out of the body of the patient this movement is indicated by numerals (E) and (F) respectively. With the help of these movements it is possible for the laparoscope camera to be precisely focused at any precise triangulated point in the body cavity in the X, Y, Z planes respectively.

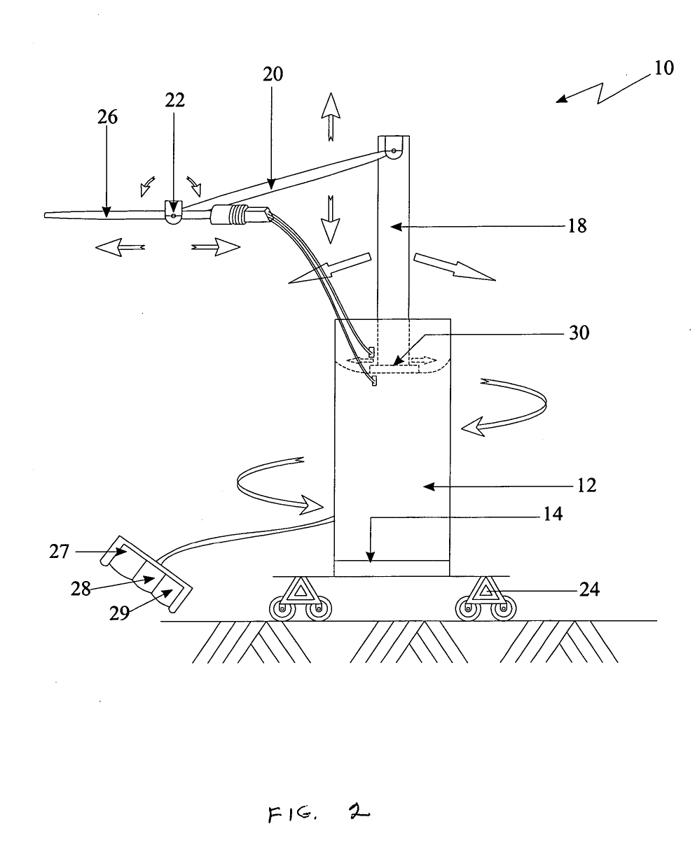

FIG. 2 illustrates the laparoscopic control equipment. A control equipment (10), for holding the laparoscopic probe and moving the tip of laparoscopic probe within the body cavity of the patient, said control equipment comprises:
(i) a housing (12) mounted on a pedestal (14) having wheels;
(ii) an angularly displaceable post (18) extending from said body;
(iii) an angularly displaceable arm (20), one end being fitted in proximity to the apex of the operative top end of said post and defining a free end; and
(iv) a clamp (22) fitted to the free end of said arm adapted to hold the probe of the laparoscope in position.

The wheeled pedestal (14) helps move the control equipment to the operating table. Once the position of the control equipment (10) is fixed, the wheels (24) can be locked such that further movement is stopped. The housing (12) is adapted to be arcuately movable about a vertical axis in a first plane in its operative configuration. The housing is mounted on a pedestal (14) having wheels. The post (18) extends from said housing (12) and is angularly displaceable in a second plane, which is perpendicular to said first plane. The arm (20) is fitted in proximity to the apex of the operative top end of the post (18). The arm (20) is angularly displaceable in a third plane, which is mutually perpendicular to said first and second planes and defining a free end. The clamp (22) is fitted to the free end and holds the probe of a laparoscope in position. Plurality of foot operated means (27), (28) and (29) are provided which helps in moving the laparoscopic probe in all directions so that the tip of the laparoscopic probe (26) within the body cavity will precisely focus and catch on camera the diseased part to be operated upon. The housing (12) of the control equipment (10), the post (18) extending therefrom and the arm (20) are in aligned engagement with each other and are angled such that they are placed and move in planes, which are mutually perpendicular to each other.

The arcuate movement of the housing (12) of the control equipment (10) about a vertical axis in its operative configuration facilitates the movement of the laparoscopic probe (26) in and out of the body cavity of the patient corresponding to the movement along the z-axis. The post (18) can be displaced 60° in the forward and backward direction about the cradle support joint (30) wherein a linear movement of the joint (30) provides an angular movement to the post (18). This angular forward and backward displacement of the post (18) is translated into a sideways displacement of the tip of the laparoscopic probe (26), which corresponds to the displacement of the viewing tip of the laparoscopic probe along the x-axis. The arm (20) can be displaced 60° in upward and downward direction with reference to the post (18). This upward and downward displacement of the arm (20) is translated into a downward and upward displacement of the tip of the laparoscopic probe (26), which corresponds to the displacement of the viewing tip of the laparoscopic probe along the y-axis. The movement of the housing (12), post (18) and arm (20) facilitates the movement of the laparoscopic probe (26) in planes which are substantially perpendicular to each other namely the X, Y and Z plane thus facilitating the exact positioning of the tip of laparoscopic probe such as to locate and precisely position the tip of the laparoscopic probe (26) at a triangulated point in the X, Y and Z planes.

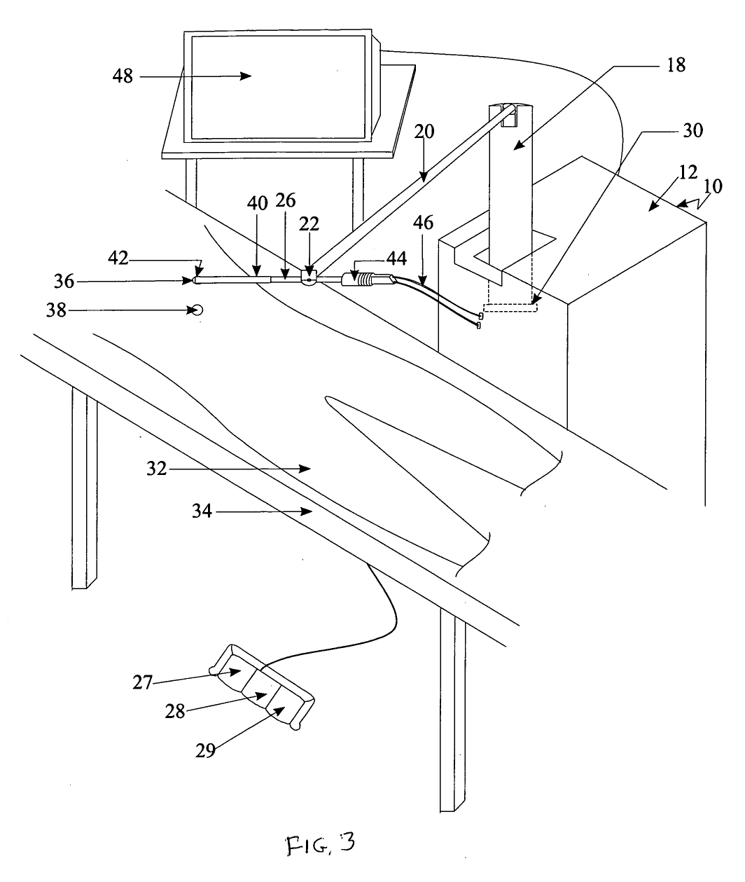

FIG. 3 illustrates an overview of the entire system in its operative configuration. The patient (32) is made to lie on the operating table (34). After administering an anesthetic, incisions (36) and (38) are made on the body of the patient (32). The exact location of the incisions depends on the type of surgery being performed. The control equipment (10) is placed at a distance such that the laparoscopic probe (26) can easily be introduced into the body cavity of the patient (32).

Through the incision (36) the trocar cannula (40) is introduced into the body cavity of the patient (32). Once the trocar cannula (40) is introduced into the body of the patient (32) through the incision (36) it is not removed until the surgery is completed. The size of the incision (36) is smaller than the diameter of the trocar cannula (40), hence the portion of the patients body abutting the trocar cannula (40) presses against the walls of the trocar cannula (40) providing an air tight sealing. The point where the human body abuts the trocar cannula forms a fulcrum point (42).

See-sawing movement of the trocar cannula (40) takes place at the fulcrum point (42). The post (18) is displaced 60° in the forward and backward direction about the cradle support joint (30) wherein a linear movement of the joint (30) provides an angular displacement to the post (18). This angular forward and backward displacement of the post (18) is translated into a sideways displacement of the laparoscopic probe (26), which corresponds to the displacement of the laparoscopic probe along the x-axis. Due to the presence of the fulcrum point (42) when the clamped portion of the laparoscopic probe (26) moves to the right the tip of the laparoscopic probe (26) moves to the left. The arm (20) is displaced 60° in upward and downward direction with reference to the post (18). This upward and downward displacement of the arm (20)) is translated into an upward and downward displacement of the laparoscopic probe (26), which corresponds to the displacement of the laparoscopic probe along the y-axis. Due to the presence of the fulcrum point (42) when the clamped portion of the laparoscopic probe (26) moves up the tip of the laparoscopic probe moves down.

The laparoscopic camera (44) captures the images observed by the lens. A wired connection (46) between the camera (44) and electronic circuitry housed within the housing (12) of the control equipment (14) helps transfer the stored images to the electronic circuitry, which in turn is displayed on a monitor (48).

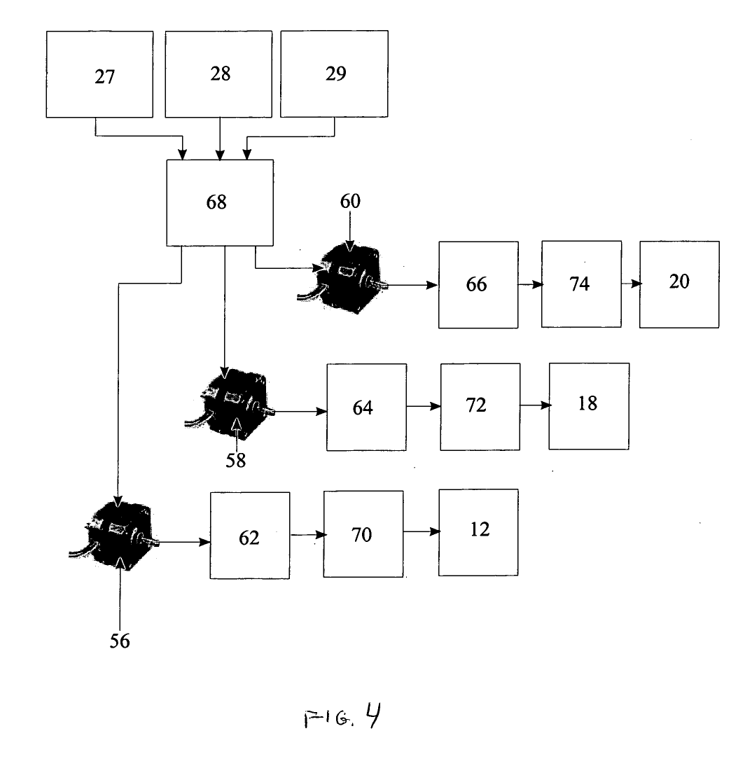

FIG. 4 illustrates a schematic block diagram of the components used to displace the housing, post and the arm of the control equipment. The housing of the control equipment houses the transformers, DC stepper motors (56), (58) and (60), discrete drive mechanisms (62), (64) and (66), electronic circuitry (68) and the like components, which facilitate the movement of the housing (12), the post (18) and the arm (20) of the control equipment. Typical embodiment of the control equipment inay be the following. Suitably stepped down DC transformer, typically 24V DC transformer is used to power the DC stepper motors (56), (58) and (60) respectively. The motors (56), (58) and (60) are used to drive said discrete drive mechanism (62), (64) and (66) respectively. The DC stepper motors (56), (58) and (60) are controlled through foot operated means (27), (28), and (29) respectively, said foot operated means (27), (28) and (29) are adapted to be connected to said motors via a programmable logic controller or microcontroller (68). A first motor (52) is connected to the housing through a first drive mechanism (62). The first drive mechanism (62) can be a gear mechanism. The first drive mechanism displaces a first link (70), which arcuately moves the housing (12). A second motor (54) is connected to the post (18) through a second drive mechanism (64) and a second link (72) The second drive mechanism (64) displaces the second link (72), which angularly displace the post (18) in a forward and backward direction. A third motor (56) is connected to the arm through a third drive mechanism (66) and a third link (74). The third drive mechanism (66) displaces the third link (74), which angularly displaces the arm (20) in an upward and downward direction. Other drive mechanisms can be envisaged, for instance a movement of the post and the arm may be controlled by a screw connected to the motor stub.

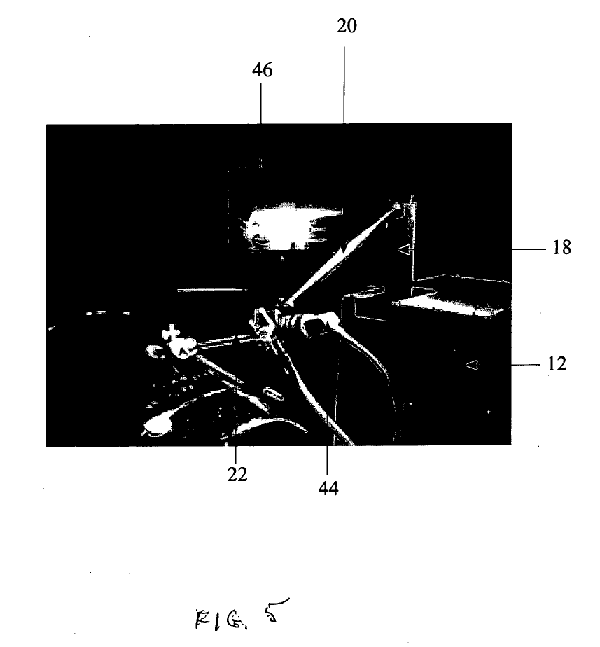

FIG. 5 illustrates a real time image of the use of the control equipment. The body (12) of the laparoscopic equipment (10) is brought sufficiently close to the operating table and locked in position. The housing (12) the post (18) and the arm (20) of the control equipment (10) are adjusted such that the surgeon can easily introduce the laparoscopic probe into the body cavity of the patient. The images captured by the laparoscopic camera (44) can be viewed on the monitor (48). Observing the image on the monitor the surgeon performs the necessary corrective procedure.

While considerable emphasis has been placed herein on the various components of the preferred embodiment, it will be appreciated that many alterations can be made and that many modifications can be made in the preferred embodiment without departing from the principles of the invention. These and other changes in the preferred embodiment as well as other embodiments of the invention will be apparent to those skilled in the art from the disclosure herein, whereby it is to be distinctly understood that the foregoing descriptive matter is to be interpreted merely as illustrative of the invention and not as a limitation.

1. A control equipment, for holding a laparoscopic probe and precisely positioning the tip of said probe in its operative configuration within a body cavity said control equipment comprising:
(i) a housing adapted to be arcuately movable about a vertical axis in a first plane in its operative configuration;
(ii) a post extending from said housing, angularly displaceable in a second plane being perpendicular to said first plane;
(iii) an arm fitted in proximity to the apex of the operative top end of said post, said arm being angularly displaceable in a third plane being mutually perpendicular to said first and second planes and defining a free end;
(iv) a clamp adapted to be fitted to said free end and adapted to hold the probe of a laparoscope in position;
(v) discrete drive mechanisms adapted to displace said housing, post and arm in respective first, second and third planes; and
(vi)control means adapted to drive each of said discrete drive mechanism means to position the tip of said probe in its operative configuration within a body cavity.

2. A control equipment as claimed in claim 1, wherein control means are a plurality of discrete foot operated means adapted to drive each of said discrete drive mechanisms.

3. A control equipment as claimed in claim 1, wherein said housing is adapted to be rotatably fixed to a pedestal having wheels for moving said control equipment.

4. A control equipment as claimed in claim 3, wherein said discrete drive mechanism means are housed within said housing.

5. A control equipment as claimed in claim 1, wherein a first fitment means is provided between said post and said housing in the form of a cradle support joint.

6. A control means as claimed in claim 1, wherein a second fitment means is provided between said arm and said post in the form of a swivel joint.

7. A method for moving the tip of a laparoscopic probe within a body cavity using a control equipment having an arcuately movable housing, a post extending therefrom an arm in proximity to the apex of said post in its operative configuration and a clamp fitted to one end of the arm for the purpose of holding a laparoscope, said method comprising:
(a) inserting said laparoscopic probe through a cannula into a body cavity said junction of body cavity and the cannula acting as a fulcrum point;
(b) moving said probe in and out of a body cavity by arcuately moving housing of said control equipment about its vertical axis in its operative configuration in a first plane;
(c) moving the tip said laparoscopic probe operatively to the right and left at said fulcrum point by angularly displacing said post along an operatively vertical axis in a second plane perpendicular to said first plane;
(d) moving the tip of said laparoscopic probe operatively up and down at said fulcrum point by swiveling said arm along an axis defined by a plane parallel to a horizontal axis and being mutually perpendicular to said first and second planes; and
(e) independently controlling said movements of the laparoscopic probe in said planes by means of remotely located control means to accurately position the viewing tip of said laparoscopic probe at a desired point within said body cavity.
